(19) United States
(12) Patent Application Publication
(10) Pub. No.: US 2007/0095346 A1
(43) Pub. Date: May 3, 2007

(54) Breathing regulatory aid

(76) Inventor: Jon Gunnar Jonsson, Port Moody (CA)

Correspondence Address: JON GUNNAR JONSSON, A-1 DENTAL LAB., #211-2922 GLEN DRIVE, COQUITLAM, BC, V3B-2P5, CA

(21) Appl. No.: 11/541,510

(22) Filed: Oct. 2, 2006

(51) Int. Cl. A61M 15/08

(52) U.S. Cl. 128/203.23

(57) ABSTRACT

The breathing regulatory aid is a self-help tool, for people to become aware of their breathing and to train good breathing habits, with restful stops in the breathing cycle. The invention is a new easy to carry tool, that makes use of conscious thumb movements, to prompt and regulate the more unconscious breathing movements of the body. The device provides a platform for simple pendulum like movement of the thumb and the device has two elevated markers, one towards each end of the said pendulum movement. People can use the tool discreetly to help the mind stay focused on the task of breathing right. By using this device the awareness of the ebb and flow of the breathing cycle is increased. As a training tool it helps people to replace a short shallow breathing habit, with a fuller breathing pattern and include stop moments in the breathing cycle.

### BACKGROUND OF THE INVENTION

The field of this invention relates to personal care products, and training tools for developing healthy breathing habits. According to medical science, relaxed full breathing is part of a healthy condition. People under stress often develop a short shallow breathing habit, with little stops between the inhaling and the exhaling. Stress directly affects the quality of breathing and restricts it. On the other hand conscious full breathing is a known way to counteract and correct stressed conditions in the body and the mind. To correct and to be conscious of a bad breathing habit is a real problem for people. The main reason is that breathing is mostly an unconscious and an automatic movement of the body. The mind is therefore quick to forget breathing as something to be consciously occupied with, and usually switches to other thoughts after only few breaths. This breathing regulatory aid uses rhythmic finger movements to keep the mind from wandering. That is a similar concept as using Prayer Beads for keeping the mind focus on praying. The breathing regulatory aid provides the platform for the thumb to move in a rhythmic way and to become a prompter and regulator, for fuller breathing and restful stops in a breathing cycle.

### SUMMARY OF THE INVENTION

The intended use of this new device. The breathing regulatory aid is a central tool for a breathing technique which teaches people, to give full attention to 4-12 breaths, few times throughout the day. Another intended use is to break up short shallow breathing pattern when it occurs and replace it with full breathing and 2 restful stops prominent in the breathing cycle. The aim of the exercise is to use breathing to reduce stress. The thumb is placed over the breathing regulatory aid, which acts as a platform for the thumb to pivot between the markers. The users learn to let the thumb lead the breathing. The first marker is the minus point and the second marker is the plus point. People move the thumb gently towards the second marker and inhale and stop the thumb there and the breathing for a restful second. Then people move the thumb towards the first marker, and follow by exhaling and stopping the thumb there and the breathing for another restful moment, and so it continues.

This device is designed for the thumb, but any other finger could replace the thumb. An object of the new invention is to provide an easy to carry breathing aid that is close to the person throughout the day. Also the tool and the small finger movements are to be discreet in design and execution, so that people can use it without others noticing. The object is to make it very simple to focus on the breathing and to correct breathing patterns whenever needed. It is also an object of the present invention to help people to discover and include stop seconds in the breathing cycle. Very few people are aware of the restful stop moment that is at the end of the inhaling, and also at the end of the exhaling. The two markers on this device are designed to draw full attention to these restful stops in the breathing cycle, by providing stop points in the pendulum move of the thumb.

To increase the stimuli for the thumb, the markers are like elevated buttons, or dome shaped bumps, to give a definite touch sensation when the thumb stops on top of these markers. The breathing regulatory aid helps people to include the restful stop moments into the breathing cycle. That is very useful in developing a relaxed breathing rhythm. Breathing is a rhythmic movement and the stop moments are a factor that calms the breathing rhythm, so people do not hyperventilate by breathing rapidly in and out.

The stop moments are also an important factor in breaking up a short stressful breathing pattern, where there are no stop moments between the shallow inhaling and exhaling.

A characteristic feature of this breathing regulatory aid and the novelty is, that it provides an easy to carry device or a platform for the conscious thumb movements, that can be used to prompt the more unconscious breathing movements of the body. This breathing regulatory aid relies on the fact that these thumb movements are fully conscious movements. People therefore decide with their mind to do these easy thumb movements, and to use them as a prompter to stay focused on that task of breathing right.

By using this aid people can consciously regulate the breathing and incorporate restful stops into the breathing cycle and correct a short shallow breathing habit. The mind is willing to stay focused longer, with the thumb movements on the device prompting the breathing, than with just breathing alone. With this tool people can quickly breathe 10-30 breaths, before their mind wanders. That is a great improvement compared with trying to remember breathing without that help. During the use of this device people focus on rhythmic and relaxed full breathing. That in turn is known to give people a feeling of inner control and rest, even under stressful circumstances.

As a self-help device, the breathing regulatory aid would be most useful as a surface item on objects that are close to the hands throughout the day. It could be added onto objects as a sticker or it could be incorporated into the surface design of objects in the manufacturing stage. It would be placed on the objects in such a way that the thumb can easily pivot between the two markers on the elliptical platform.

The breathing regulatory aid could be placed on surfaces like; a glue on sticker, a briefcase-handle, a barrel of a pen, a back of a cell phone, a side of a music player, a back of a TV-remote-control, a back of a car-remote-control, a key ring, a jewellery item, a back of a pocket-knife, a lighter, a side of a laptop computer or a computer mouse, a side of a wristband, a side of a wristwatch-strap, a back of a wallet, a side of a arm rest, a belt buckle, or on a designed finger-clip that goes over the index finger, to name a few. The breathing regulatory aid would be easy to include in the surface details, of hand held objects made from plastic or rubber, silicone, metal, wood or leather for example.

The design of the breathing regulatory aid is shown on a flat surface in the drawings, but could be applied to a curved surface like the barrel of a pen and the design would curve with the surface of the object it is applied to.

It is to be understood that the description and embodiments set forth above can be modified, without departing from the true spirit of this invention, which is an easy to carry breathing regulatory aid, that provides the platform for conscious thumb movements and touch sensations at stop markers and the aid utilizes that to prompt and regulate the more unconscious breathing cycle, and in so doing the aid helps people to stay focused on the task of breathing right.

### BRIEF DESCRIPTION OF DRAWINGS

FIG. 1 is a top plan view showing my new device.

FIG. 2 is a front elevational view of FIG. 1; the rear elevation view is a mirror image.

FIG. 3 is a bottom plan view of FIG. 1.

FIG. 4 is a front right side elevational view of FIG. 1; the left side elevation view is a mirror image.

FIG. 5 is a pictorial view showing the device as applied to an instrument of writing, being shown as environment.

FIG. 6 is a pictorial view showing the device, as applied to a designed finger-clip as environment, shown resting on an index finger.

FIG. 7 is a top plan view showing the device as applied to a fingerclip, shown as environment.

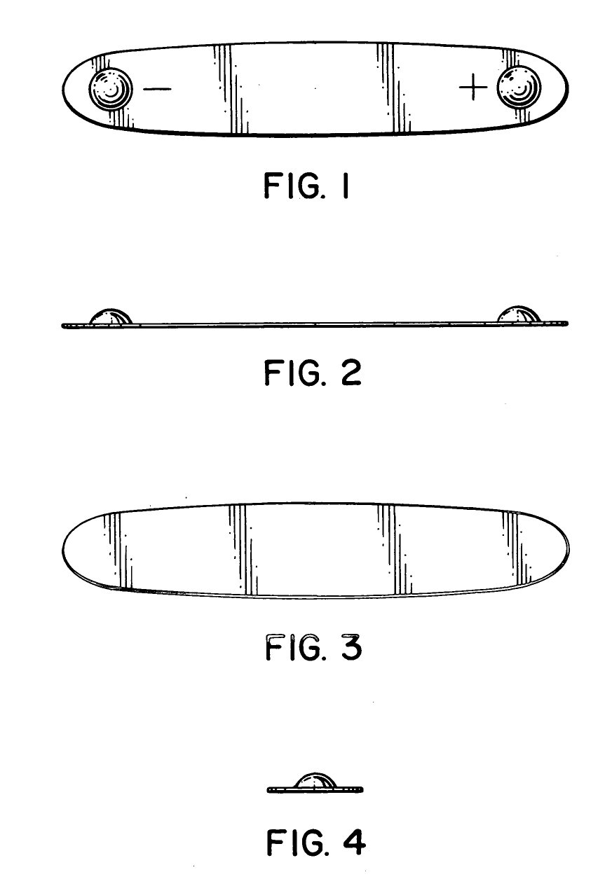

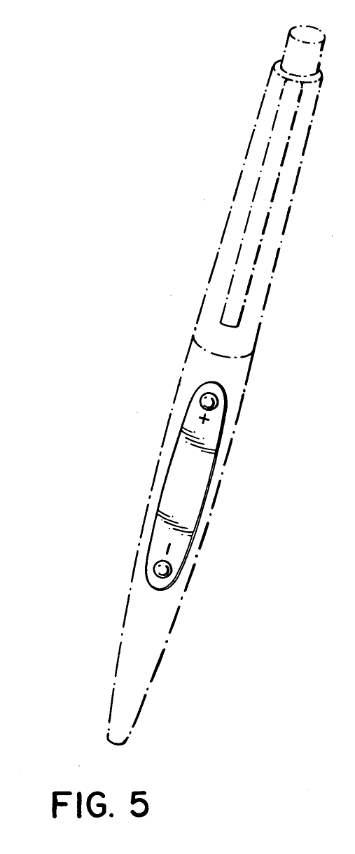

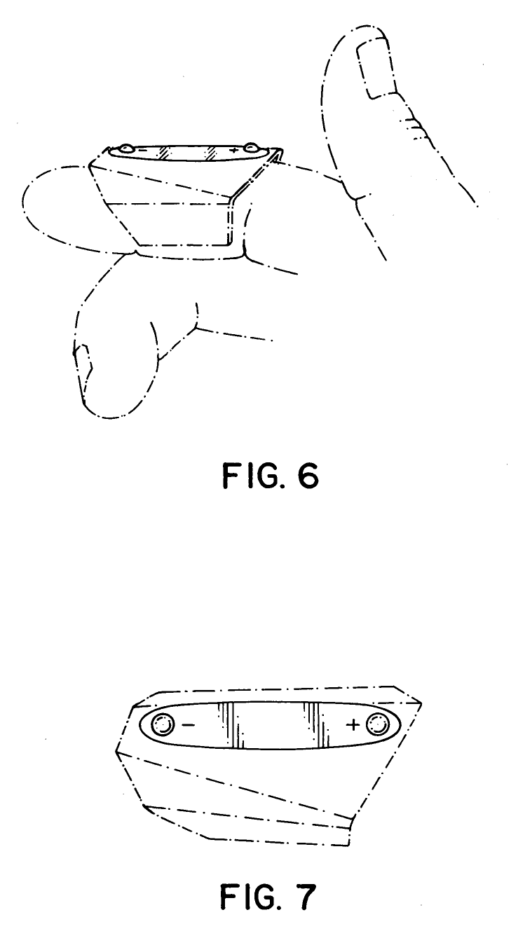

I claim:

1. A breathing regulatory aid comprising a first marker and a second marker placed in fixed relation to each other as reference points to coordinate movement of a user's digit from the first marker to the second marker during inhaling and from the second marker to the first marker during exhaling.

2. The breathing regulatory aid of claim 1, in which at least one of the first and second markers is a bump with respect to a planar surface between the first and second markers.

3. The breathing regulatory aid of claim 1, in which the first and second markers are bumps with respect to a planar surface between the first and second markers.

4. The breathing regulatory aid of claim 1, in which the first marker is placed a user's thumb pivot distance away from the second marker such that a user's thumb can pivot from being on the first marker for exhaling to being on the second marker for inhaling.

5. The breathing regulatory aid of claim 1, in which the first marker and the second marker are mounted on a platform.

6. The breathing regulatory aid of claim 4, in which the platform has an adhesive backing for mounting to an object such as a cell phone.

7. The breathing regulatory aid of claim 4, in which the platform is mounted on a pen.

8. The breathing regulatory aid of claim 1, in which the first marker and the second marker are mounted on a top surface of a platform having side flanges for retaining the aid on an index finger of the user.

9. The breathing regulatory aid of claim 4, in which the platform is elliptical in shape.

10. The breathing regulatory aid of claim 2, in which:
a) the first and second markers are substantially button shaped bumps;
b) the first marker is placed a user's thumb pivot distance away from the second marker such that a user's thumb can pivot from being on the first marker for exhaling to being on the second marker for inhaling;
c) the first marker and the second marker are mounted on a top surface of a platform having side flanges for retaining the aid on an index finger of the user.
